(19) United States
(12) Patent Application Publication
(10) Pub. No.: US 2004/0152975 A1
(43) Pub. Date: Aug. 5, 2004

(54) Image registration

(76) Inventor: Ira Blevis, Zichron-Yaacov (IL)

Correspondence Address: William H. Dippert, Esq., c/o Reed Smith LLP, 29th Floor, 599 Lexington Avenue, New York, NY, 10022-7650, US

(21) Appl. No.: 10/354,926

(22) Filed: Jan. 30, 2003

(51) Int. Cl. A61B 6/00

(52) U.S. Cl. 600/427

(57) ABSTRACT

Apparatus for providing image registration of a target, comprising a gamma camera that acquires a nuclear image of said target, an optical imager in a fixed or known relationship with respect to said gamma camera that acquires an optical image of said target and an image registrator operative to receive and register said nuclear image and said optical image.

### FIELD OF THE INVENTION

The present invention relates to nuclear image registration.

### BACKGROUND OF THE INVENTION

Image registration of two images, such as X-ray and nuclear images of internal human anatomy, provide visualization of cancerous nodes and surrounding organ structure. However, to make an initial incision that properly encompasses the cancerous nodes, the surgeon often relies upon estimating the node location in relation to the skin. The resultant incision, for example, is often imprecise and/or of improper length or configuration, requiring modification after the surgical area is exposed so the cancerous nodes can be properly removed.

Incision location and configuration are extremely important. For example, an incision that improperly traverses a joint may result in long recuperation, poor joint function and/or loss of extremity use. Recognizing this problem, the surgeon may place radiopaque markers on the skin, for example fixed wires or paint that appear in the registered radiographic image.

Balan, et al. in WO 00/75691 A1, the disclosure of which is incorporated herein by reference, present an apparatus for providing scans from a gamma camera and tomographic imager of a target.

Lemchen in U.S. Pat. No. 6,081,739 demonstrates a method for using video surface scanning, 3D surface reconstruction and X-ray for visualizing the position of teeth versus the lips, cheeks and facial features.

### SUMMARY OF THE INVENTION

An aspect of some embodiments of the present invention relates to registering a nuclear image of a target on or within a tissue, for example a tumor and/or its sentinel nodes, with an optical image of the skin of the target, to provide a registered image that guides a surgeon in planning and executing an incision. An incision based upon a registered image from nuclear and optical images encompasses the tumor and nodes without undue extension and/or damage to adjacent tissue.

The registered image includes, for example, specific anatomical landmarks such as breast position, bone protrusion or position, hair, birthmarks, scars and/or tattoos to aid the surgeon in planning the incision. Additionally or alternatively, the skin is marked with markers that show in the optical image and are registered with the nuclear image to provide a registered image with markings that aid in planning the incision.

Optionally, the skin is marked with radiopaque markings that show up as fiduciary markings in the optical image and as radioactive markings in the nuclear image. In an embodiment of the invention, the fiduciary markings on the optical image are aligned with the radioactive markers on the nuclear image by an operator who superimposes the two images to provide a registered image. Additionally or alternatively, registration of the two images is provided by an image registrator that aligns the radioactive markings in the nuclear image with the fiduciary markings in the optical image.

In an exemplary embodiment of the present invention, an optical imager, for example a photographic digital camera that provides at least still views, and a gamma camera, are mounted on a gantry. The optical imager is focused on the external surface of a patient and precisely aligned with the gamma camera to automatically provide a registered image. In some embodiments, the camera is not mounted upon the gantry but upon an arm that moves in measurable relationship to the gantry.

Optionally, the gantry includes an X-ray imager that registers an X-ray image along with the nuclear and optical registered images, to provide, for example, a registered image that provides further information on the target. In some embodiments, an X-ray image comprises a planer radiographic image. In some embodiments, an X-ray image comprises a digital planer radiographic image. Additionally or alternatively, an X-ray image comprises an X-ray tomographic image.

In an embodiment of the present invention, the registered image including an optical image, and optionally a nuclear and/or X-ray image, is provided as a picture, for example as a hard copy. Additionally or alternatively, the registered image is projected by a projector onto a projection site, for example, located in the operating theater.

In an embodiment of the invention, the registered image is shown on a viewing station, for example, located in the operating theater. In an embodiment of the invention, controls are provided on the viewing station to allow an operator to outline an incision directly on the viewing station registered image. Optionally, controls are provided to vary the relative intensity of image components, for example anatomic features, so the tumor and/or incision, for example, are seen clearly and distracting aspects of the registered image removed.

Optionally, controls are provided to vary the relative intensity of radioactive image components, so the tumor, tumor nodes and/or radioactive markers are seen clearly in relation to other aspects of the registered image. Additionally or alternatively, relative intensity of registered image components are varied using a software-based program that is operational by the operator and/or automatic. Optionally, a picture of the registered image shown on the viewing station is produced and placed, for example, on an image viewing apparatus in the operating theater. Additionally or alternatively, the image on the viewing station is projected by a projector onto a projection site, for example, located in the operating theater.

In an embodiment of the present invention, a projector projects the registered image, including an optical image, and optionally a nuclear and/or X-ray image, onto the subject's corresponding anatomy. An operator manipulates the projection so that the optical image of the registered image lines up with the subject's skin anatomy. In an embodiment of the invention, the image is manipulated so that only the planned incision is visible and the surgeon makes an incision using the projected incision as a guide.

In an embodiment of the invention, the registered image, whether a picture, a viewing station image and/or a projector image, is sectored into multiple component including landmarks that appear at different stages of the surgery. Landmarks include, for example, skin markings, a drawn incision and/or anatomic structures such as nerves, cartilage or vascular tissue. As the surgery progresses to different anatomical sectors or depths within the target, the surgeon uses the component images as a visual map of anatomic structures, landmarks and/or other features to be encountered.

There is thus provided an apparatus for providing image registration of a target, comprising a gamma camera that acquires a nuclear image of said target, an optical imager in a fixed or known relationship with respect to said gamma camera that acquires an optical image of said target and an image registrator operative to receive and register said nuclear image and said optical image.

In an exemplary embodiment, said gamma camera and said optical imager are mounted on a gantry. Optionally, the optical imager comprises a digital camera or a video camera.

In an exemplary embodiment, said registrator receives images of said target from an X-ray imager and registers an X-ray image with said optical image. Optionally, said X-ray imager is fixed or moveable in a measurable relationship with respect to said gamma camera. Alternatively or additionally, said X-ray imager is mounted on a gantry together with said nuclear camera and gamma camera and said optical imager.

In an exemplary embodiment, the X-ray imager provides a planer X-ray image. Alternatively or additionally the X-ray imager provides a planer digital X-ray image and/or a tomographic image.

In an exemplary embodiment, the optical imager images optically visible markers on said target, wherein said image register an image generated by said nuclear imager with an optical image including said optical markers. Optionally the optical imager includes an X-ray imager that registers an image received by said X-ray imager with an optical image including said optical markers.

In an exemplary embodiment, said apparatus comprises a display that displays a registered image. Optionally, said display comprises an image highlighter control operative to change the relative emphasis of said nuclear image and said optical image in said registered image, responsive to an operator input.

In an exemplary embodiment, the image highlighter is provided on said data display that changes the relative emphasis of one or more of said X-ray image, nuclear image and optical image in said registered image. In a further exemplary embodiment, said apparatus includes an X-ray imager, wherein a tissue emphasizor is provided on said display that changes the relative emphasis of one or more tissues in said registered image.

Optionally, said emphasizor includes an image demarcator control operative to provide demarcation of an incision on said registered image. Optionally, said demarcator includes a demarcator emphasizor that changes the relative emphasis of said incision demarcation on said registered image.

In an exemplary embodiment, a radioactive component expressor is included in said data display that changes the relative emphasis of one or more of said tumor and tumor nodes in said registered image. Optionally, said radioactive component expressor changes the relative emphasis of one or more of said radioactive markers, tumor and tumor nodes in said registered image.

In an exemplary embodiment, the image registrator provides one or more printed registered images. Alternatively or additionally, the image registrator includes a plurality of registered image viewing stations. Optionally, the apparatus includes a data storage device, wherein the registered image is saved on retrievable storage media.

There is thus provides a method for registering nuclear and optical images of a target, comprising obtaining a nuclear image of a target, obtaining an optical image of a target and registering said nuclear image and said optical image. Optionally, said method includes obtaining an X-ray image of said target and registering it with said nuclear image and said optical image.

In an exemplary embodiment, optical markers are placed on said target and recording said target with said optical imager. Optionally, said optical markers that are radioactive on said target and recording said target with said nuclear imager.

In a further exemplary embodiment, a planned tissue incision is marked on said registered image and said registered image is output on a viewing station image. Optionally a hard copy of said registered image is put out. Further, an incision on said target is marked using said registered image as an incision guide.

There is thus provided a method for image registration, comprising providing a composite image of the internal and external structure of a portion of a subject, adjusting the relative intensities of the internal and external components of the image and displaying the composite image. In an exemplary embodiment, the external image comprises an optical image. In an exemplary embodiment, the internal image comprises a gamma camera image. Optionally, said displaying includes displaying on a monitor. Optionally said displaying includes providing a hard copy.

### BRIEF DESCRIPTION OF THE DRAWINGS

Exemplary non-limiting embodiments of the invention will be described with reference to the following description of embodiments in conjunction with the figures. Identical structures, elements or parts which appear in more than one figure are preferably labeled with a same or similar number in all the figures in which they appear.

FIG. 1 is a representation of a radiographic and nuclear image registration system, according to an embodiment of the present invention;

FIG. 2 is a representation of a projected registered image, according to an embodiment of the present invention;

FIG. 3 is a representation of a gantry for obtaining radiographic and nuclear images registered with an X-ray image, according to an embodiment of the present invention;

FIG. 4 is a representation of optical and nuclear images registered with an X-ray image, according to an embodiment of the present invention; and

FIG. 5 is a representation of a registered image that has been sectored into component images, according to an embodiment of the present invention.

### DETAILED DESCRIPTION OF EMBODIMENTS

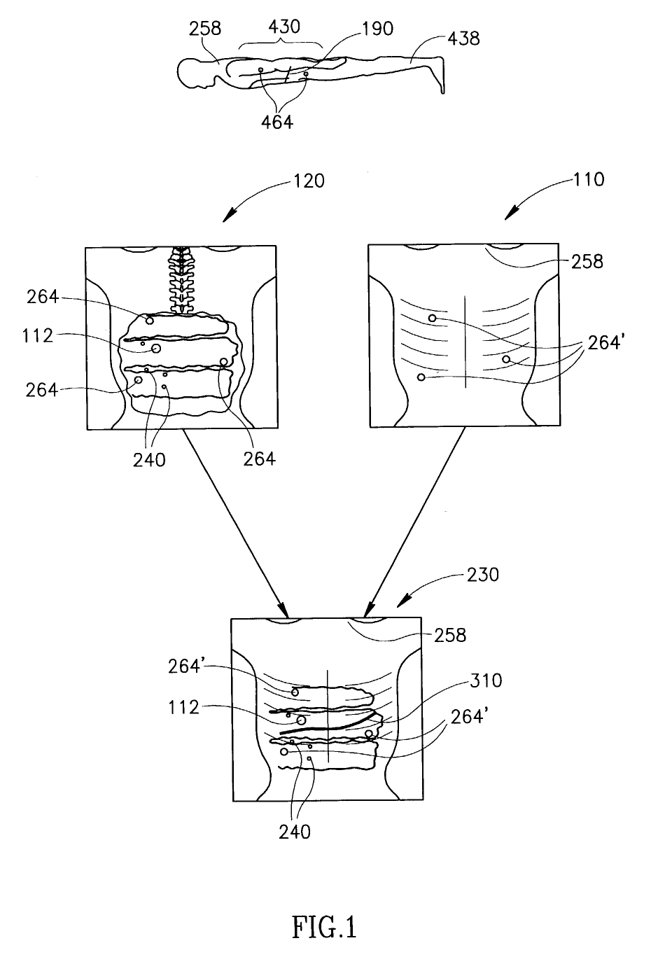

FIG. 1 is a representation of a nuclear image 120, including a tumor 112 and nodes 240 that have absorbed a nuclear medicine agent, and an optical skin image 110 of a target 430 on a subject 438. Nuclear image 120 and optical skin image 110 are registered to provide a registered image 230 that includes an image of tumor 112 and nodes 240 and surface features of skin image 110. By utilizing guideposts from optical skin image 110 in respect to image of tumor 112 and nodes 240, the surgeon draws an incision tracing image on registered image 230 corresponding to the planned physical incision.

In an exemplary embodiment, guideposts that aid in locating incision tracing image 310 consist of skin anatomy, for example a bone protrusion 258. Additionally or alternatively, landmarks comprise, for example, hair, birthmarks, scars, surface contours and/or tattoos.

In an embodiment of the present invention, skin markers 464 are placed on target 430 to provide skin marker images 264 in optical skin image 110 that appear in registered image 230. The surgeon makes an incision 190 in relation to skin markings 464 by viewing skin marking images 264 on registered image 230. To accommodate a possible long delay between placement of skin markers 464 and the onset of surgery, skin markers 464 may be indelible, for example, for about 12 hours.

Optionally, skin markers 464 comprise a radiopaque paint that, for example, appears in nuclear image 120 as markers 264 and in optical skin image 110 as skin markers 264′. In an embodiment of the invention, an operator superimposes a picture of optical image 120 with skin markers 264′ on a picture of nuclear image 120 with markers 264 to provide registered image 230 with skin marking images 264′.

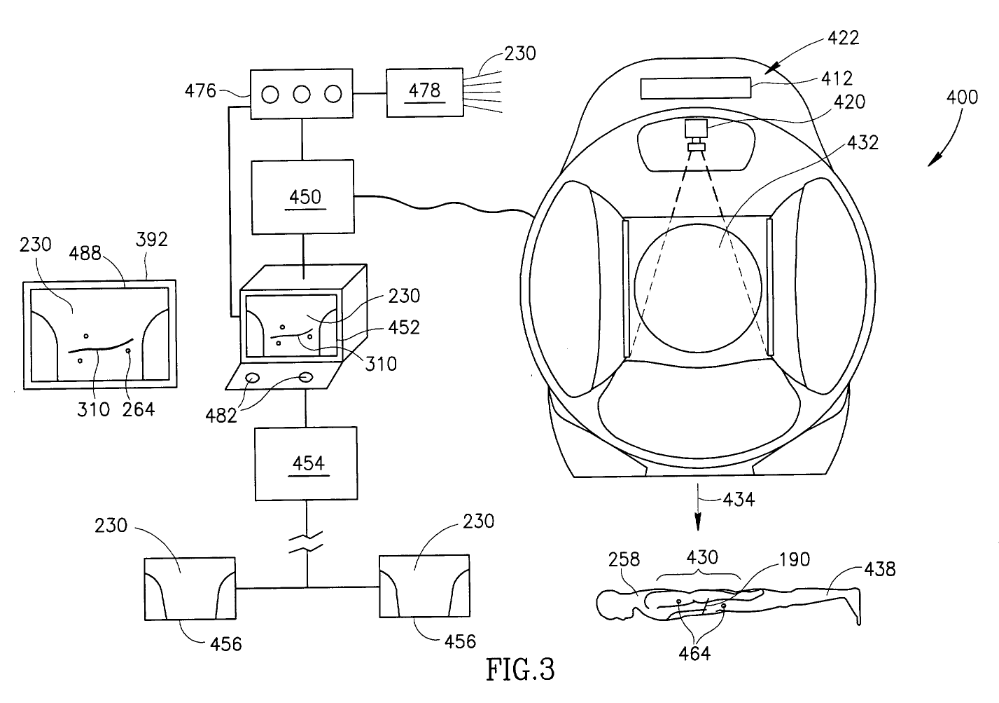

In an embodiment of the present invention, an image registrator 450 (FIG. 3) registers images 110 and 120 to provide a single picture 488 that is, for example a hard copy, of registered image 230. To provide registered image 230, for example, image registrator 450 aligns markers 264 in received nuclear image 120 with skin markers 264′ in received optical skin image 110. Additionally or alternatively, the fields of view of a gamma camera 422 and an optical imager 420 are precisely aligned, for example on a gantry 400, and received nuclear image 120 and optical skin image 110 are automatically registered by image registrator 450.

Picture 488 includes landmarks and/or skin markers 264′, corresponding to skin markings 464 that appear on target 430 and is, for example, placed on an image viewer 392 (FIG. 3) in the surgical theater. Prior to, or as surgery begins, the surgeon makes incision tracing image 310 on picture 488 that most efficiently encompasses tumor 112 and nodes 240 and uses it as a map for making incision 190.

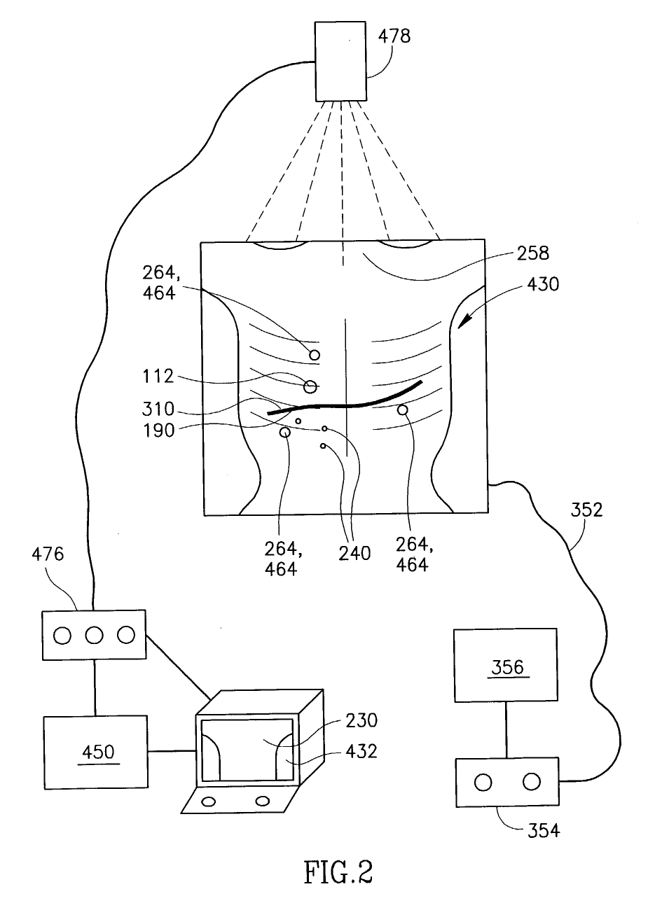

Additionally or alternatively, registered image 230 is provided on a viewing station 452, or projected as an image from a projector 478 (FIG. 2) within the surgical theater that is viewed by the surgeon and used as a map by which the surgeon cuts skin incision 190 on target 430.

Optionally, prior to donning sterile surgical garb, the surgeon manipulates controls 482 to produce incision tracing image 310 on viewing station 452. Optionally, viewing station 452 provides picture 488 and/or image from projector 478 that includes incision tracing 310.

The surgeon duplicates incision tracing image 310 on subject 438 as incision 190, using the location of skin marker images 264 and/or anatomic landmarks as guideposts. Optionally, viewing station 452 is programmable so an operator can program it to show multiple images, each image including, for example, incision tracing 310, along with one or more anatomic landmarks, for example, nerves, cartilage, tumor or vascular tissue, as will be explained below. These images provide the surgeon with maps of the target 430 as he cuts incision 190 deeper to different levels of tissue.

FIG. 2 is a representation of projector 478 that projects registered image 230, comprising registered images 110 and 120 onto a viewing area, for example target 430. Orientation controls 476 are used to change the position, orientation and/or size of projected registered image 230 so that skin marker images 264 properly align with skin markings 464 and registered image 230 is properly aligned with target 430.

Registered image 230, for example, is shown on screen 452 and controls 482, for example, are used to draw incision tracing image 310 on screen 452 so that it appears on projected registered image 230. Registered image 230, including incision tracing image 310, is projected onto target 430 and manipulated, for example with orientation and size controls 476 to line up with target 430. At the onset of surgery, the surgeon cuts physical incision 190 directly into the patient skin, following projected incision tracing image 310. Additionally or alternatively, the surgeon draws a guide on the patient skin following projected incision tracing image 310, turns off projected image 230 and makes incision 190 along the guide.

In an embodiment of the present invention, a non-sterile assistant operates orientation controls 476 to change position, orientation and/or size of projected registered image 230. Additionally or alternatively, sterile covers on controls 476 allow the surgeon to manipulate controls 476 while in the sterile operating theater. Optionally, controls 476 are operated by surgeon voice activation in the operating theater. Additionally or alternatively, controls 476 are non-sterile foot pedals accessible by the surgeon in the operating theater.

In an embodiment of the invention, registered image 230 is aligned on target 430 and then skin image 110 is de-emphasized in projected registered image 230, leaving nuclear image 120 projection and/or skin markers image 264′ projected on target 430. The surgeon physically makes incision 190 directly on target 430 to encompass tumor 112 and nodes 240.

Additionally or alternatively, skin image 110 is emphasized in registered image 230 and projected with skin markers 264′ and the surgeon makes incision 190 on target 430 according to skin markers 264′. Additionally or alternatively, components of registered image 230, for example anatomic structures, are emphasized or de-emphasized as will be explained below.

In an embodiment of the present invention, a laparoscopic probe 352 with a control panel 354 is used to take, for example, a biopsy from target 430. Using skin marker images 264 on viewing station image 356 and skin markings 464 on patient target 430, the surgeon can accurately position probe 352 to take a biopsy.

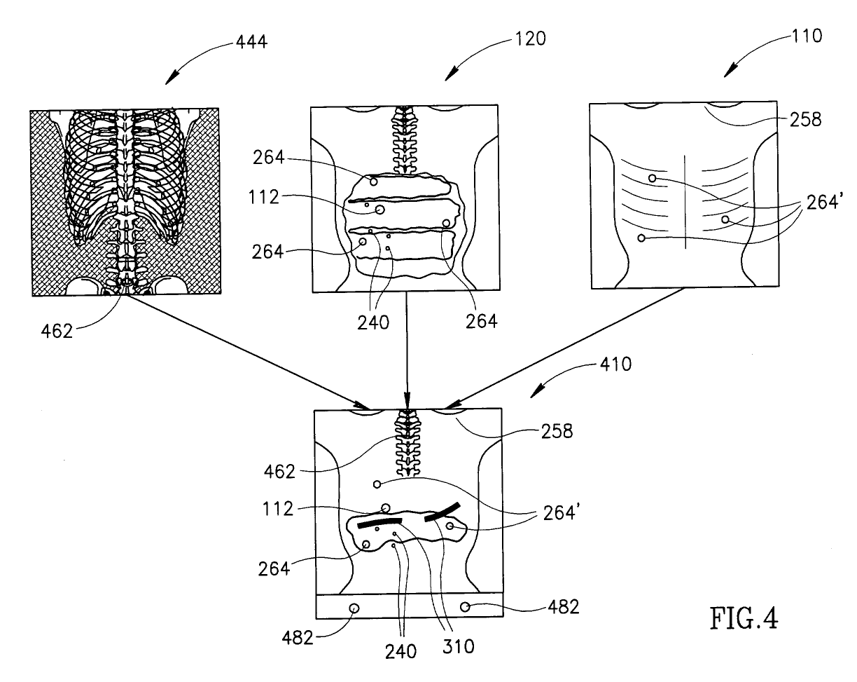

FIG. 4 is a representation of optical image 110 and nuclear image 120 registered with an X-ray image 444 to provide a registered image 410 on viewing screen 452. Optionally, image 410 can be manipulated with controls 482 to demonstrate individual images 110, 120 and/or 444 and/or multiple images that are overlaid in any combination as required by the surgeon.

Emphasizing different images, 110, 120 and/or 444 of registered image 410 allows the surgeon, for example, to accurately estimate the tissue planes in which tumor 112 and/or individual nodes 240 are located. For example, one or more sentinel nodes 240 may be on a plane below a specific organ, such as a spine 462 on X-ray image 444. By emphasizing bony spine image 462 in registered image 410, while de-emphasizing optical image 110 and/or nuclear image 120, the surgeon clarifies the location of sentinel nodes 240. Considering this knowledge, he revises his initial incision tracing image 310, for example, to be two small incisions on either side of spine 462, rather than a single incision encompassing spine 462.

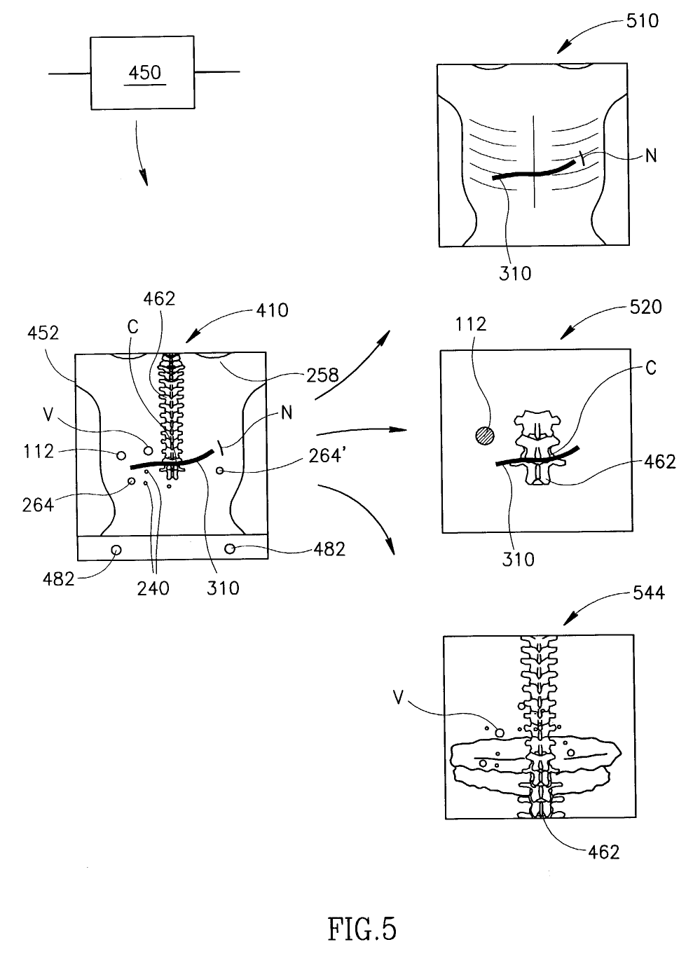

In an embodiment of the invention, as shown in FIG. 5, image registrator 450, produces registered image 410 on viewing screen 452 and the operator uses controls 482 to highlight specific anatomic structures and/or regions, for example, a nerve N, an area of cartilage C vascular tissue V and/or tissue that has absorbed a nuclear medicine agent, for example, tumor 112. The operator, for example, may produce an image 510 with emphasized incision 310 and nerve N and de-emphasized optical image 110. Additionally or alternatively, image 510 is produced using a software-based program that is operational by the operator and/or an automatic program. Using image 510 at the outset of surgery, the surgeon for example, avoids over-extending incision 310 and possibly severing nerve N.

Additionally or alternatively, the operator may produce an image 520 with emphasized incision 310, cartilage area C and tumor 112. The surgeon, for example, may call for display of image 520 to provide an accurate map of structures as he deepens incision 310 into cartilage area C.

Additionally or alternatively, the operator may produce an image 544 with emphasized spine 462 and tumor nodes 240 that have absorbed a nuclear medicine agent. The surgeon, for example, may call for display of image 544 as he begins deep dissection of nodes 240.

Images 510, 520 and/or 544, for example, are displayed, for example, according to the surgeon's wishes throughout the surgical procedure, as pictures 488 on a picture viewing apparatus, as images on viewing station 452 and/or as projections from image projector 420. Additionally or alternatively, images 510, 520, 544 or other images composed from registered image 410, are shown on multiple viewing stations 456, located for example throughout a hospital, that can be viewed by qualified professionals. This provides an opportunity, for example, for multi-disciplinary participation in planning and/or observation of surgery of target 430.

FIG. 3 is a representation of gantry 400 comprising gamma camera 422 and optical imager 420. Optionally, gantry 400 includes X-ray imager 412. Gantry 400, for example, moves in direction 434 until a field of view 432 of subject 438 can be taken of target 430. For further details of gantry 400, please refer to Balan, et al., WO 00/75691 A1.

In an embodiment of the invention, gantry 400 takes images with gamma camera 422, optical imager 420 and optionally X-ray imager 412 and sends these images to image registrator 450. Image registrator 450 registers the images to produce picture 488, an image for projector 478 and/or an image on viewing station 452. Optionally, output 452 is connected to a storage device 454 that records registered image 230 on appropriate storage media that can be retrieved to be viewed on multiple viewers 456, for example, located in different hospital areas and/or departments, allowing intra-disciplinary consultation over registered image 230.

In an embodiment of the present invention, X-ray imager 412 comprises a planer X-ray imager. Additionally or alternatively, X-ray imager 412 comprises a planer X-ray imager that provides digital planer X-ray images. Additionally or alternatively, X-ray imager 412 comprises an X-ray tomograph.

In an exemplary embodiment, gamma camera 422 registers an image of a radioactive nuclear medicine agent, for example Gallium-67 citrate or any other appropriate radioactive nuclear medicine agent, that has concentrated in, for example, tumor 112 and nodes 240.

Optical imager 420 is, for example, a digital camera that provides digital images that are, for example, registered in registered image 230. Additionally or alternatively, camera 420 is a video camera that provides video images that are registered in registered image 230. Additionally or alternatively, camera 420 is held separately from gantry 400 and is moveable with respect to gantry 400 in a measurable relationship to gantry 400.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

Furthermore, the terms “comprise,” “include,” “have” and their conjugates, shall mean, when used in the claims, “including but not necessarily limited to.”

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

1. An apparatus for providing image registration of a target, comprising:
(a) a gamma camera that acquires a nuclear image of said target;
(b) an optical imager in a fixed or known relationship with respect to said gamma camera that acquires an optical image of said target; and
(c) an image registrator operative to receive and register said nuclear image and said optical image.

2. Apparatus according to claim 1, wherein said gamma camera and said optical imager are mounted on a gantry.

3. Apparatus according to claim 1, wherein the optical imager comprises a digital camera.

4. Apparatus according to claim 1, wherein the optical imager comprises a video camera.

5. Apparatus according to claim 1, wherein said registrator receives images of said target from an X-ray imager and registers an X-ray image with said optical image.

6. Apparatus according to claim 5, wherein said X-ray imager is fixed or moveable in a measurable relationship with respect to said gamma camera.

7. Apparatus according to claim 5, wherein said X-ray imager is mounted on a gantry together with said nuclear camera, said gamma camera and said optical imager.

8. Apparatus according to claim 1, wherein the X-ray imager provides a planer X-ray image.

9. Apparatus according to claim 8, wherein the X-ray imager provides a planer digital X-ray image.

10. Apparatus according to claim 5, wherein the X-ray imager provides a tomographic image.

11. Apparatus according to claim 1, wherein the optical imager images optically visible markers on said target and said image registrator registers an image generated by said nuclear imager with an optical image including said optical markers.

12. Apparatus according to claim 11 and including an X-ray imager that registers an image received by said X-ray imager with an optical image including said optical markers.

13. Apparatus according to claim 1 and comprising a display that displays a registered image.

14. Apparatus according to claim 13 and comprising an image highlighter control operative to change the relative emphasis of said nuclear image and said optical image in said registered image, responsive to an operator input.

15. Apparatus according to claim 13 and including an X-ray imager, wherein an image highlighter is provided on said data display that changes the relative emphasis of one or more of said X-ray image, nuclear image and optical image in said registered image.

16. Apparatus according to claim 13 and including an X-ray imager, wherein a tissue emphasizor is provided on said display that changes the relative emphasis of one or more tissues in said registered image.

17. Apparatus according to claim 13 and comprising an image demarcator control operative to provide demarcation of an incision on said registered image.

18. Apparatus according to claim 17, wherein said demarcator includes a demarcator emphasizor that changes the relative emphasis of said incision demarcation on said registered image.

19. Apparatus according to claim 13 and including radioactively tagged tumor and tumor nodes, wherein a radioactive component expressor is included in said data display that changes the relative emphasis of one or more of said tumor and tumor nodes in said registered image.

20. Apparatus according to claim 19, wherein said radioactive component expressor changes the relative emphasis of one or more of radioactive markers, tumor and tumor nodes in said registered image.

21. Apparatus according to claim 1, wherein the image registrator provides one or more printed registered images.

22. Apparatus according to claim 1, wherein the image registrator includes a plurality of registered image viewing stations.

23. Apparatus according to claim 1 and including a data storage device, wherein the registered image is saved on retrievable storage media.

24. A method for registering nuclear and optical images of a target, comprising:
a) obtaining a nuclear image of a target;
b) obtaining an optical image of a target; and
c) registering said nuclear image and said optical image.

25. A method according to claim 24 and including obtaining an X-ray image of said target and registering it with said nuclear image and said optical image.

26. A method according to claim 24, comprising placing optical markers on said target and recording said target with said optical imager.

27. A method according to claim 24, comprising placing optical markers that are radioactive on said target and recording said target with said nuclear imager.

28. A method according to claim 24, comprising marking a planned tissue incision on said registered image.

29. A method according to claim 24, comprising outputting a viewing station image of said registered image.

30. A method according to claim 24, comprising outputting a hard copy of said registered image.

31. A method according to claim 24, comprising making an incision on said target using said registered image as an incision guide.

32. A method of image registration, comprising:
a) providing a composite image of the internal and external structure of a portion of a subject;
b) adjusting the relative intensities of the internal and external components of the image; and
c) displaying the composite image.

33. A method according to claim 32, wherein the external image comprises an optical image.

34. A method according to claim 33, wherein the internal image comprises a gamma camera image.

35. A method according to claim 32, wherein said displaying includes displaying on a monitor.

36. A method according to claim 32, wherein said displaying includes providing a hard copy.
